(19) United States
(12) Patent Application Publication
(10) Pub. No.: US 2019/0392031 A1
(43) Pub. Date: Dec. 26, 2019

(54) Storage Medium, Medical Instruction Output Method, Medical Instruction Output Apparatus and Medical Instruction Output System

(71) Applicant: KONICA MINOLTA INC., Chiyoda-ku (JP)

(72) Inventor: Eiichi AKAGI, Hachioji-shi (JP)

(73) Assignee: KONICA MINOLTA INC., Tokyo (JP)

(21) Appl. No.: 16/447,689

(22) Filed: Jun. 20, 2019

(30) Foreign Application Priority Data
Jun. 22, 2018 (JP) 2018-118370

(51) Int. Cl. G06F 17/24; G10L 15/08; G10L 15/26; G06F 3/16; G06F 3/14; G06T 11/60; G16H 30/20

(52) U.S. Cl. CPC G06F 17/241; G10L 15/08; G10L 15/26; G10L 2015/088; G06F 3/14; G06T 11/60; G16H 30/20; G06F 3/16

(57) ABSTRACT

A non-transitory computer-readable storage medium having a program stored thereon for controlling a computer to perform the following, obtaining time series sound data which includes spoken contents by a speaker; obtaining operation information combined image data or operation information combined moving image data including a medical image and image operation information which includes contents of an image operation performed on the medical image; extracting the time series sound data in a time region with high importance; extracting the operation information combined image data or the operation information combined moving image data in the time region with high importance; and outputting at least one of the time series sound data in the time region with high importance and the operation information combined image data or the operation information combined moving image data in the time region with the high importance.

### BACKGROUND

### 1. Technological Field

The present invention relates to a storage medium, a medical instruction output method, a medical instruction output apparatus and a medical instruction output system.

### 2. Description of the Related Art

In a typical medical diagnosis, a medical specialist specializing in a specific disease may examine a patient while looking at a medical image imaged by X-ray imaging in a hospital. During an examination, the medical specialist may give instructions regarding treatment after the examination. Such instructions given by the medical specialist may be recorded as comments in clinical records or instruction documents made by the medical specialist himself. Alternatively, a nurse who heard the instructions may record the instructions as comments.

The example in which an emergency case occurs is considered. When an emergency case occurs in a hospital, and the patient may have a cerebral infraction, a special diagnosis is necessary immediately. However, the medical doctor who can make a suitable diagnosis may not be in the hospital when such medical doctor is at home at nighttime, for example. In such case, medical images may be transmitted to the medical specialist (medical doctor who can make a suitable diagnosis) at home, and the diagnosis may be requested to the medical specialist. The medical specialist in a remote location from the hospital uses a smartphone or tablet to perform diagnosis through an internet connection or a telephone connection. Usually, the result of the diagnosis needs to be informed to the medical doctor in the hospital immediately, and there is no time to write a report while performing the diagnosis. As a result, the instruction is made orally using the telephone.

However, instructing orally lacks accuracy due to error in listening or difference in understanding. In order to prevent such problems, for example, there is a method to record instructions made orally in a written form using the technique described in Japanese Patent Application Laid-Open Publication No. 2018-73067. However, such technique merely records all of the words said by the medical specialist and it may not be easy to determine how to instruct the patient specifically.

### SUMMARY

The object of the present invention is to accurately notify items regarding the medical instruction by the medical specialist without errors.

To achieve at least one of the abovementioned objects, according to an aspect of the present invention, a storage medium reflecting one aspect of the present invention is described, the non-transitory computer-readable storage medium having a program stored thereon for controlling a computer to perform: obtaining time series sound data which includes spoken contents by a speaker; obtaining operation information combined image data or operation information combined moving image data including a medical image and image operation information which includes contents of an image operation performed on the medical image; extracting the time series sound data in a time region with high importance from the time series sound data; extracting the operation information combined image data or the operation information combined moving image data in the time region with high importance from the operation information combined image data or the operation information combined moving image data; and outputting at least one of the time series sound data in the time region with high importance and the operation information combined image data or the operation information combined moving image data in the time region with the high importance.

According to another aspect of the present invention, a medical instruction output method includes: obtaining time series sound data which includes spoken contents by a speaker; obtaining operation information combined image data or operation information combined moving image data including a medical image and image operation information which includes contents of an image operation performed on the medical image; extracting the time series sound data in a time region with high importance from the time series sound data; extracting the operation information combined image data or the operation information combined moving image data in the time region with high importance from the operation information combined image data or the operation information combined moving image data; and outputting at least one of the time series sound data in the time region with high importance and the operation information combined image data or the operation information combined moving image data in the time region with the high importance.

According to another aspect of the present invention, a medical instruction output apparatus includes: a hardware processor which performs, obtaining time series sound data which includes spoken contents by a speaker; obtaining operation information combined image data or operation information combined moving image data including a medical image and image operation information which includes contents of an image operation performed on the medical image;

extracting the time series sound data in a time region with high importance from the time series sound data; extracting the operation information combined image data or the operation information combined moving image data in the time region with high importance from the operation information combined image data or the operation information combined moving image data; and outputting at least one of the time series sound data in the time region with high importance and the operation information combined image data or the operation information combined moving image data in the time region with the high importance.

According to another aspect of the present invention, a medical instruction output system includes: a server including a hardware processor which performs, obtaining time series sound data which includes spoken contents by a speaker; obtaining operation information combined image data or operation information combined moving image data including a medical image and image operation information which includes contents of an image operation performed on the medical image; extracting the time series sound data in a time region with high importance from the time series sound data; and extracting the operation information combined image data or the operation information combined moving image data in the time region with high importance from the operation information combined image data or the operation information combined moving image data; and a terminal which displays at least one of the time series sound data in the time region with high importance and the operation information combined image data or the operation information combined moving image data in the time region with the high importance.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1A shows a medical instruction output apparatus according to the present embodiment.

FIG. 1B shows a block diagram showing a medical instruction output system according to the present embodiment.

FIG. 2A is a diagram showing a procedure flow of a viewer process in a mobile terminal.

FIG. 2B is a diagram showing a procedure flow of a sound process in the mobile terminal.

FIG. 2C is a diagram showing a procedure flow by a server.

FIG. 2D shows a list screen and a view screen.

FIG. 3 is a diagram showing a procedure flow by a hospital terminal.

FIG. 4 is a diagram showing an operation flow by a user.

FIG. 5A is a screen display example showing a text display version.

FIG. 5B is a screen display example showing a sound reproduction version.

FIG. 6 shows a confirmation mark example of a message showing an instruction information display.

### DETAILED DESCRIPTION OF EMBODIMENTS

Hereinafter, one or more embodiments of the present invention will be described with reference to the drawings. The embodiments described below include various technically preferable limitations to implement the present invention. However, the scope of the invention is not limited to the disclosed embodiments.

Embodiments of the present invention are described below with reference to the diagrams. However, the scope of the present invention is not limited to the illustrated examples.

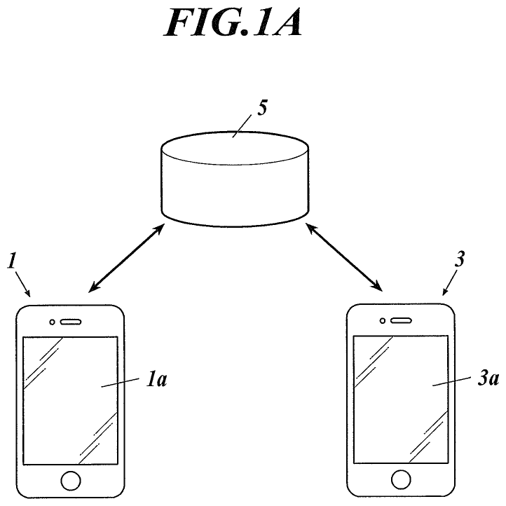

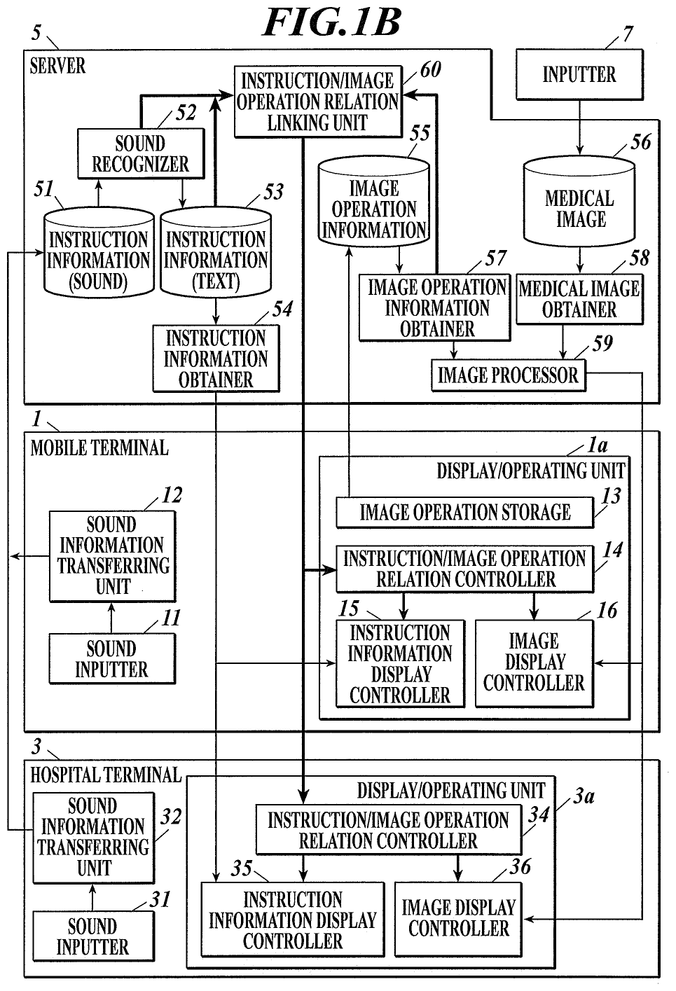

FIG. 1A is a medical instruction output apparatus according to an embodiment of the present invention. FIG. 1B is a block diagram showing a medical instruction output system according to an embodiment of the present invention.

As shown in FIG. 1A, a mobile terminal 1 is a terminal which a medical specialist of various diseases carries to prepare for emergency patients, and a hospital terminal 3 is a terminal provided in a hospital to save and view patient information of the emergency patient. For example, the mobile terminal 1 and the hospital terminal 3 are touch panel type portable terminals as shown in FIG. 1A, and each include a display/operating unit 1a and a display/operating unit 3a respectively. Examples of the display device included as the display/operating units 1a, 3a include a liquid crystal display (LCD), a cathode-ray tube (CRT) display, an organic electronic luminescence (EL) display, a nonorganic EL display, and a plasma display.

The configuration of the mobile terminal 1 and the hospital terminal 3 is not limited to the above, and can be any terminal which can display patient information.

For example, the server 5 includes a central processing unit (CPU), a read only memory (ROM), and a random access memory (RAM).

The server 5 includes a storage function to store programs and various data, and therefore is provided with a hard disk drive (HDD) or a nonvolatile semiconductor memory.

Next, FIG. 1B is described.

The mobile terminal 1 includes a sound inputter 11, a sound information transferring unit 12, an image operation storage 13, an instruction/image operation relation controller 14, an instruction information display controller 15, and an image display controller 16.

The sound inputter 11 obtains all of the detectable sound data including words emitted by a speaker (spoken contents) and stores the sound data. Preferably, the accuracy of detecting the sound is a degree so that the spoken contents can be detected completely, but the present embodiment is not limited to the above.

The sound information transferring unit 12 transfers sound data obtained by the sound inputter 11 to a sound recognizer 52 in a server 5.

The image operation storage 13 includes the function to store contents of the image operation performed as image operation information (shown with image operation information 55 in FIG. 1B) when the image operation is performed on a medical image 56. Examples of the image operation include various operations such as, adding an annotation a, expansion/reduction of the medical image 56, gradation process, panning, measurement (process to measure length of lesion site), and attaching comments by text entry.

Alternatively, an image operation inputter (not shown) which includes the function to obtain the image operation information 55 and the letter inputter (not shown) to input letters can be provided separately from the image operation storage 13 and the image operation storage 13 may include the function to obtain the image operation information 55 as described above.

The image operation storage 13 transfers the obtained image operation information 55 to an image operation information obtainer 57 of the later-described server 5.

The present embodiment is described on the assumption that the medical image 56 is a still image (for example, X-ray image), but this may be a moving image (for example, moving images of CT or MRI related to kinetic analysis).

The instruction/image operation relation controller 14 includes the function to transfer to the instruction information display controller 15 and the image display controller 16 information linking the instruction information (sound) 51 and the image operation information 55 or the instruction information (text) 53 and the image operation information 55. The above information is linked to each other by an instruction/image operation relation linking unit 60 in the server 5.

The instruction/image operation relation controller 14 controls the display/operating unit 1a to display the linked information at the same time and not separately. However, the instruction information (sound) 51 and the image operation information 55 or the instruction information (text) 53 and the image operation information 55 do not always have to be displayed together. Only the instruction information 51, 53 or only the image operation information 55 can be displayed.

Such instruction information display controller 15, image display controller 16, instruction information (sound) 51, instruction information (text) 53, image operation information 55, instruction/image operation relation linking unit 60 are described in detail later.

The instruction information display controller 15 displays the instruction information (text) 53 obtained by the instruction information obtainer 54 of the server 5 (later-described) on the display/operating unit 1a.

Based on the control from the instruction/image operation relation controller 14, the instruction information display controller 15 displays the instruction information (sound) 51 or the instruction information (text) 53 on the display/operating unit 1a.

The function of the instruction information display controller 15 may be included in the instruction/image operation relation controller 14, and in this case, the instruction information display controller 15 can be omitted.

The image display controller 16 displays on the display/operating unit 1a the medical image 56 obtained by the image processor 59 of the server 5 (later-described) and the image operation information 55.

The image display controller 16 displays on the display/operating unit 1a the image operation information 55 based on the control from the instruction/image operation relation controller 14.

The function of the image display controller 16 can be included in the instruction/image operation relation controller 14, and in this case, the image display controller 16 can be omitted.

The hospital terminal 3 includes a sound inputter 31, sound information transferring unit 32, instruction/image operation relation controller 34, instruction information display controller 35, and an image display controller 36.

The sound inputter 31 includes the same function as the sound inputter 11 of the mobile terminal 1.

The sound information transferring unit 32 includes the function similar to the sound information transferring unit 12 of the mobile terminal 1.

The instruction/image operation relation controller 34 includes the function similar to the instruction/image operation relation controller 14 of the mobile terminal 1.

The instruction information display controller 35 includes the function similar to the instruction information display controller 15 of the mobile terminal 1.

The image display controller 36 includes the function similar to the image display controller 16 of the mobile terminal 1.

The server 5 includes a sound recognizer 52, instruction information obtainer 54, image operation information obtainer 57, medical image obtainer 58, image processor 59, and an instruction/image operation relation linking unit 60.

The sound recognizer 52 converts the spoken contents in the sound data obtained by the sound inputter 11 in the mobile terminal 1 and the sound inputter 31 in the hospital terminal 3 to text. The spoken contents in the sound data are shown in the instruction information (sound) in FIG. 1B. The sound recognizer 52 can change the instruction information (sound) 51 to text in real time simultaneously with the instruction information (sound) 51 being obtained. The method to change the information to text is not limited, and any well-known method to change sound to text can be used.

The sound recognizer 52 can be configured to be able to recognize that the information is output by a different person for each terminal used in the sound input. That is, it is possible to discriminate that the instruction information (sound) 51 obtained by the sound inputter 11 in the mobile terminal 1 shows information output by the user using the mobile terminal 1, and the instruction information (sound) 51 obtained by the sound inputter 31 in the hospital terminal 3 is information output by the user using the hospital terminal 3.

The method to discriminate the terminal is not limited to the above-description, and for example, this can be determined according to the various features of the obtained sound.

After converting the sound data to text, the sound recognizer 52 outputs the above as the instruction information (text) 53 and transfers the information to the instruction information obtainer 54.

The instruction information obtainer 54 transfers the instruction information (text) 53 transferred by the sound recognizer 52 to the instruction information display controller 15 in the mobile terminal 1 and the instruction information display controller 35 in the hospital terminal 3.

The image operation information obtainer 57 transfers the image operation information 55 transferred by the above-described image operation storage 13 to the image processor 59.

The medical image obtainer 58 transfers the medical image 56 obtained by the inputter 7 (for example, various modalities generating the medical image 56 and the other apparatuses storing the medical image 56) to the image processor 59.

The image processor 59 compares the medical image 56 transferred by the medical image obtainer 58 with the image operation information 55 transferred by the image operation information obtainer 57, and the medical image 56 reflecting the image operation information 55 is generated.

The image processor 59 transfers the generated medical image 56 to the image display controller 16 in the mobile terminal 1 and the image display controller 36 in the hospital terminal 3.

In addition to providing the image processor 59 in the server 5, the image processor 59 may be provided in the mobile terminal 1 or the hospital terminal 3. In this case, the medical image 56 is transferred to the mobile terminal 1 or the hospital terminal 3 in advance, and when the image operation information 55 is generated, the image operation information 55 may be transferred in real time to each terminal, and the medical image 56 reflecting the image operation information 55 may be generated in the image processor 59 each time and displayed. In this case, the burden of the image process in the mobile terminal 1 and the hospital terminal 3 becomes larger, but the image does not need to be transferred each time. Therefore, the image can be displayed in real time even in an environment with a narrow network band.

The instruction/image operation relation linking unit 60 includes the function to store the instruction information (sound) 51 and the image operation information 55 or the instruction information (text) 53 and the image operation information 55 linked in a time series.

Here, “linked in a time series” means stored with the time axis matching.

The embodiment of the present invention is described according to various procedure flows and operation flows.

The present embodiment assumes an example in which an emergency patient appears when the medical specialist is outside (including at home). The medical specialist at home carries the mobile terminal 1, there is the hospital terminal 3 storing the patient information in the hospital, and a doctor or nurse who is not a medical specialist uses the hospital terminal 3.

The server 5 is provided in the hospital or on a cloud, and the mobile terminal 1 carried by the medical specialist at home communicates with the hospital terminal 3 used by the medical doctor or the nurse in the hospital through the server 5.

According to the present embodiment as described above, a dedicated terminal employing the program, system, and method of the present invention is used, but the present invention is not limited to the above. Any terminal can be used, an application executing the program, system, and method of the present invention may be downloaded and used.

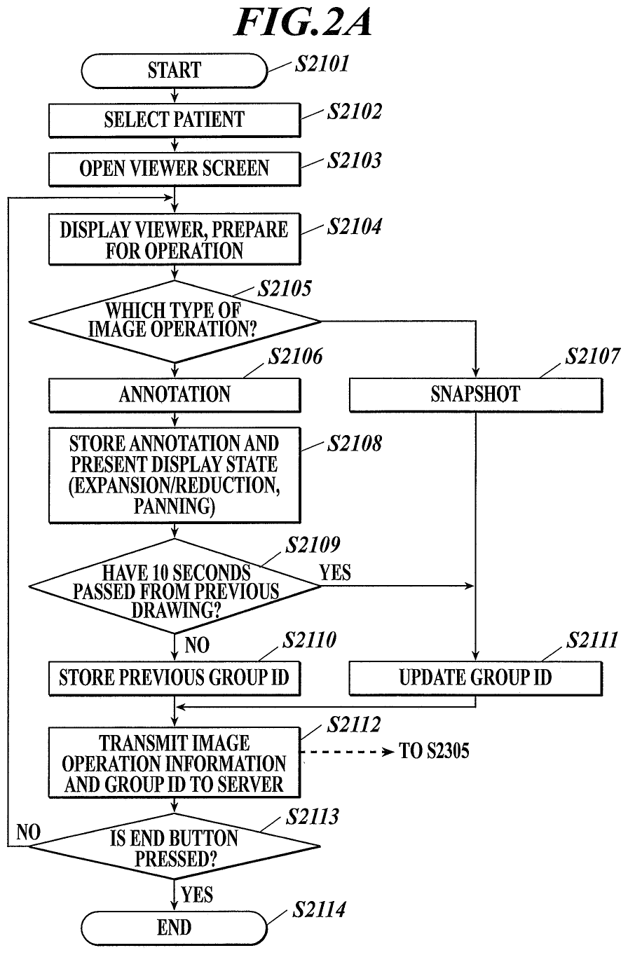

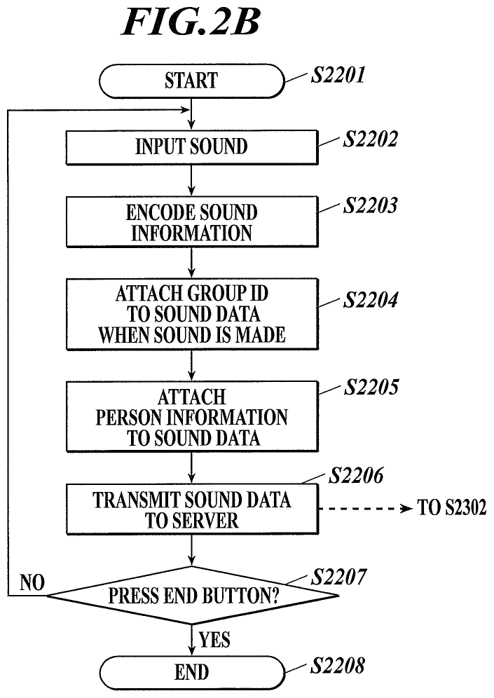

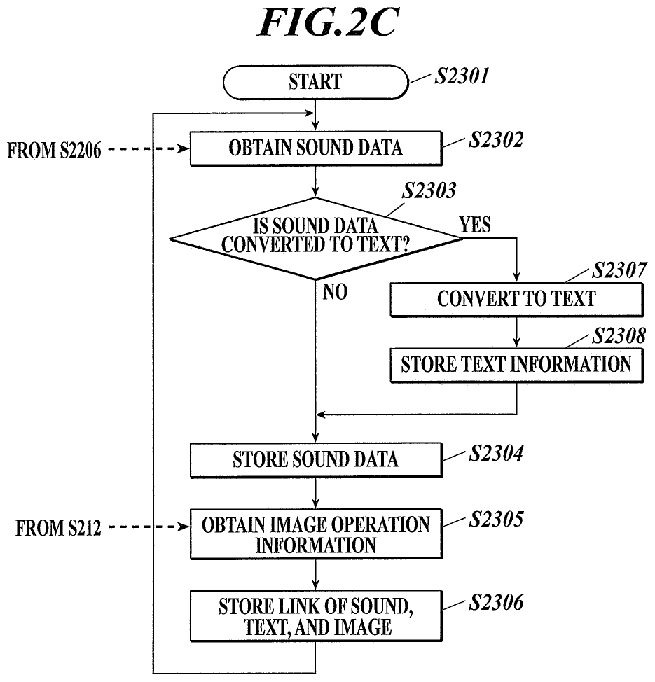

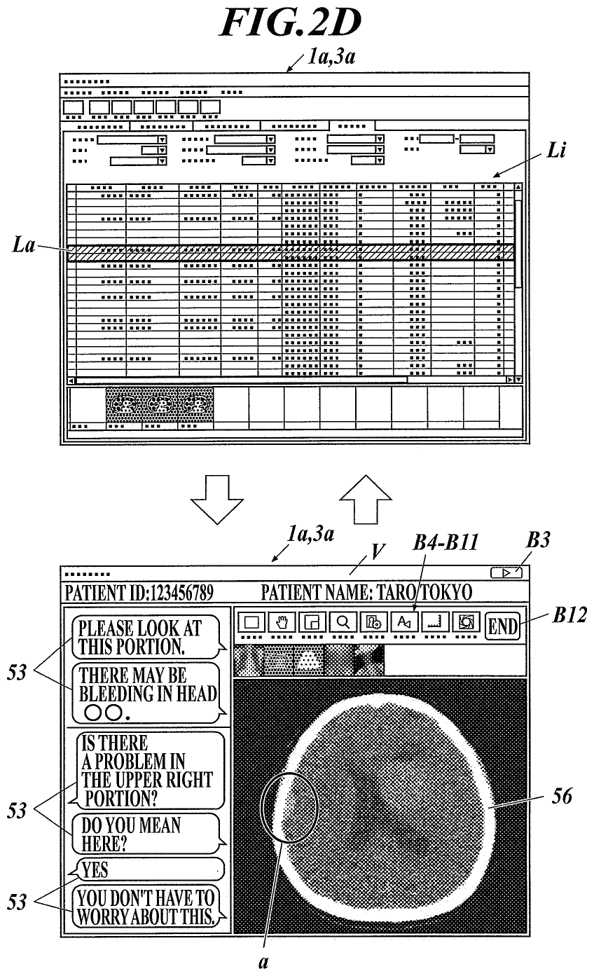

FIG. 2A to FIG. 2D show procedure flows between the mobile terminal 1 and the server 5 and screen examples. FIG. 2A is an example showing the procedure flow of the viewer process in the mobile terminal 1. FIG. 2B is an example showing the procedure flow of the sound process in the mobile terminal 1. FIG. 2C is an example showing the procedure flow of the server 5. FIG. 2D is an example of a list screen Li and a viewer screen V.

The procedure flow of the viewer process is described according to FIG. 2A. The procedure here applies to the method to obtain the image operation information 55 showing the operation performed on the medical image 56 in the mobile terminal 1.

The viewer process starts in step S2101. The medical specialist starts the process by pressing start (not shown) on the mobile terminal 1.

Next, the patient examined in step S2102 is selected. For example, line La showing the patient to be examined on the list screen Li as shown in the upper side of FIG. 2D is touched, pressed, or double clicked to select the patient.

When the patient is selected in step S2102, the viewer screen V is opened in step S2103. The viewer screen V is displayed as shown on the bottom side of FIG. 2D, and the examination image of the patient (medical image 56) and a group of various buttons B3 to B12 are displayed.

Next, in step S2104, the viewer display and preparation for operation is performed. The viewer display/operation button (not shown) is pressed and the group of buttons which are necessary to perform the image operation is displayed to set the state in the prepared state.

When in the prepared state with the viewer screen V opened, this step can be omitted.

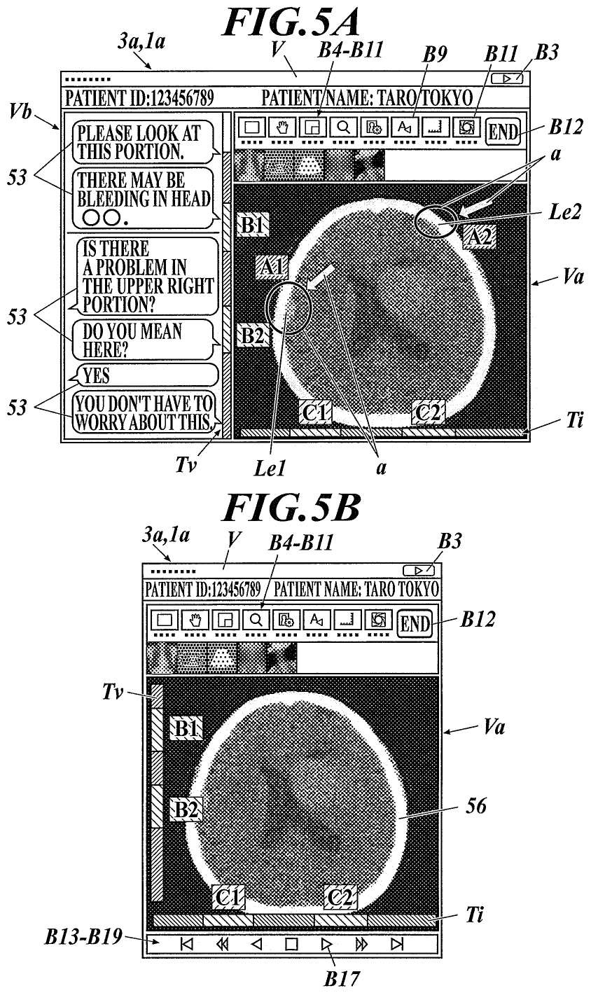

Next, in step S2105, the type of image operation selected by the user is determined. The present embodiment assumes adding the annotation a and performing the snapshot as the image operation. First, when the user selects adding the annotation a, the user presses the annotation button B9 (see FIG. 5A). With this, the process advances to step S2106.

In step S2106, the medical specialist adds the annotation a as the image operation on the medical image 56.

At the same time as adding the annotation a, the information of the added annotation a (image operation information 55) is stored in the image operation storage 13, and the group ID is attached. This is step S2108. In step S2106, as soon as the annotation a is added, the process automatically advances to step S2108.

If a predetermined amount of time has not elapsed from the previous point that the annotation a is added when the annotation a is added in step S2106, that is, the image operation information 55 is stored in step S2108 (step S2109, No), a group ID added in step S2106 is stored. This is to be step S2110.

When it is the first time that the annotation a is added, it is determined to be No in step S2109.

Next, the process automatically advances to step S2112, and the image operation information 55 stored in step S2106 and the group ID are transmitted to the server 5.

According to the present embodiment, the elapsed time used in the judgement in step S2109 is 10 seconds. Any amount of time can be set as the elapsed time used here, and alternatively, the user including the medical specialist carrying the mobile terminal 1 can set the elapsed time in advance.

After the image operation information 55 and the group ID are transmitted to the server 5 in step S2112, if the end button B12, etc. is pressed in step S2113 (Yes), the process advances to step S2114 and the viewer process ends. When the end button B12 is not pressed in step S2113 (No), the process returns to step S2104 and the process progresses in the above-described order.

As described above, when the second annotation a is added within 10 seconds from adding the first annotation a, the same group ID is attached to the two annotations a, and the same is applied for the third annotation a and after.

The image operations other than adding the annotation a include expansion and reduction. The expansion and reduction are also provided with a group ID as the image operation and the group ID is stored.

If the annotation a is added in step S2106, that is, when the image operation information 55 is stored in step S2108 after 10 seconds passed from the point that the previous annotation a is added (Yes in step S2109), the group ID is updated. This is to be step S2111.

After the group ID is updated in step S2111, the process advances to step S2112, and the image operation information 55 and the group ID are transmitted to the server 5. Then, similar to the above, the process advances to step S2112 and after.

In step S2105 (determination of type of image operation), the example in which it is determined that the user selected snapshot is described. When the user presses a snapshot button B11, the image operation information 55 before pressing the snapshot button B11 is stored, and the group ID is updated and attached to the image operation information 55 (step S2111). That is, the snapshot is a function in which the user is able to update the group ID at any timing.

All of the image operation information 55 from when the previous occasion that the group ID is attached to when the snapshot button B11 is pressed can be stored by attaching the same group ID. Alternatively, image operation information 55 within a certain time duration before pressing the snapshot button B11 can be stored by attaching the same group ID.

The image operation storage 13 attaches the group ID and transmits the image operation information 55 to the server 5.

The process described above is the procedure flow of the viewer process. Next, the procedure flow of the sound process is described with reference to FIG. 2B. The sound process is performed in both the mobile terminal 1 and the hospital terminal 3.

First, as step S2201, the sound process starts. According to the present invention, all of the sound data in a time series including the spoken contents by the speaker and sound other than those by the speaker from the start to the end can be obtained.

Here, the “time series sound data” means the sound data accumulated along a flow of time. Hereinafter, the sound data in a time series is simply called sound data. According to the present embodiment, the details of obtaining all of the sound data are omitted, and only the spoken contents by the speaker in the sound data is obtained in the sound process flow.

All of the sound data can be obtained by an all sound data obtainer which is not shown. Therefore, according to the present embodiment, the point that the speaker starts to speak is detected and the sound process starts from the point that the speaking starts.

Next, the sound input starts as step S2202. The sound inputter 11 (sound inputter 31) inputs the spoken contents by the speaker.

Next, as step S2203, encoding of the sound information starts. The spoken contents input in step S2202 are encoded in a format such as G. 711 defined by ITU-T (International Telecommunication Union Telecommunication Standardization Sector) so that the data size is compressed to a size suitable for communication. The sound inputter 11 (sound inputter 31) or the sound information transferring unit 12 (sound information transferring unit 32) performs the encoding.

Next, as step S2204, the group ID is attached to the sound data. The group ID is attached by the sound inputter 11 (sound inputter 31) and the sound information transferring unit 12 (sound information transferring unit 32). The group ID attached to the sound data corresponds to the group ID attached to the image information in the viewer process. Therefore, the group ID attached to the sound data obtained first in the sound process is the same as (corresponds to) the group ID attached to the image operation performed first in the viewer process.

Next, as step S2205, person information is attached to the sound data with the group ID attached in step S2204.

Here, “person information is attached” means to attach information regarding who was speaking in the obtained sound data. The method to attach the person information includes a method to determine the person according to the terminal which obtained the sound data. That is, the sound data obtained by the sound inputter 11 in the mobile terminal 1 is determined to be contents spoken by the medical specialist using the mobile terminal 1, and the sound data obtained by the sound inputter 31 in the hospital terminal 3 is determined to be contents spoken by the medical doctor or the nurse in the hospital using the hospital terminal 3. However, the method to determine the person is not limited to the above, and for example, features of the voice emitted from the person (volume of voice, amplitude of waveform, wavelength, etc.) can be detected and this can be used in the determination.

Next, as step S2206, the sound data with the person information attached in step S2205 is transmitted to the server 5. The transmitting to the server 5 is performed by the sound information transferring unit 12 (sound information transferring unit 32).

After transmitting the sound data to the server 5 in step S2206, it is determined whether the end button B12 is pressed. This is step S2207.

Methods other than pressing the end button B12 can be used to determine that the process ends. When it is determined that the end button B12 is pressed (Yes in step S2207), the sound process ends. This is to be step S2208. When it is determined that the end button B12 is not pressed (No in step S2207), the process returns to step S2202, and the process is performed in the above-described order.

Typically, it is difficult to determine a break (end) of sound data. For example, when a speaker speaks, then does not speak for a certain amount of time, and then speaks again, it may be difficult to determine whether to consider the spoken contents before and after the pause as the same group or to consider that the spoken contents are a different group by determining that there is a break in the sound due to a certain amount of time passing without speaking. It may be difficult to define a clear standard for determination. Preferably, the break in the sound data is not to be the standard to update the group ID of the sound data, and the group ID of the sound data is updated together with updating the group ID of the viewer process. Alternatively, the break of the sound data can be determined individually from the viewer process using features regarding the sound data. In this case, for example, the volume of the sound and the change in the waveform can be used as the feature of the sound data.

The above is the procedure flow of the sound process. Next, the procedure flow of the sever 5 is described with reference to FIG. 2C.

First, as step S2301, the operation of the server 5 starts. Here, the operation of the server 5 starts when the sound data is transmitted in step S2206. That is, as step S2302, the sound recognizer 52 in the server 5 obtains as the instruction information (sound) 51 the sound data transferred from the sound information transferring unit 12 and the sound information transferring unit 32 of the mobile terminal 1 and the hospital terminal 3.

Next, as step S2303, it is determined whether the sound data is converted to text. This is determined by the sound recognizer 52. When it is determined that the conversion to text is not performed (No in step S2303), the process advances to step S2304. When it is determined that the conversion to text is performed (Yes in step S2303), the process advances to step S2307.

Any standard can be employed to determine whether the sound is converted to text. For example, the determination of whether to convert the sound to text may be decided in advance when the system is introduced, and such standard may always be applied. Alternatively, determination standards can be provided such as the sound data may be converted to text when the sound is obtained clearer than a certain standard, and when the sound is unclear than the certain standard, the sound data may not be converted to text.

When the process advances from step S2303 to step S2307, the sound data is converted to text as step S2307. The sound recognizer 52 converts the instruction information (sound) 51 as the sound data to instruction information (text) 53. The method of how to convert the sound to text is not limited, and any well-known sound-to-text method can be used.

The instruction information (text) 53 made in step S2307 is stored in the instruction information obtainer 54. This is to be step S2308.

When step S2303 is No or after step S2308, the process advances to step S2304.

The instruction information (sound) 51 and the instruction information (text) 53 are stored in the instruction/image operation relation linking unit 60.

Next, as step S2305, the image operation information 55 transferred from the mobile terminal 1 in the viewer process is obtained. The image operation information 55 is obtained by the image operation information obtainer 57.

After the image operation information 55 is obtained by the image operation information obtainer 57, the image operation information 55 is transferred to the image processor 59 and the instruction/image operation relation linking unit 60. Here, the instruction information (sound) 51, the instruction information (text) 53, and the image operation information 55 are linked. This is to be step S2306. The medical image 56 is combined with the image operation information 55 in this step.

The instruction/image operation relation linking unit 60 links the instruction information (sound) 51, the instruction information (text) 53, and the image operation information 55. The corresponding group ID is attached to each of the instruction information (sound) 51 and the image operation information 55. The instruction information (text) 53 originates from the instruction information (sound) 51 and therefore, the group ID attached to the instruction information (text) 53 corresponds to the image operation information 55.

Here, “linking” means the instruction information (sound) 51 and the image operation information 55 or the instruction information (text) 53 and the image operation information 55, all of which attached with the corresponding group ID, are stored corresponded as one set.

The medical image 56 is combined with the image operation information 55 according to the process described below. First, the medical image 56 is input with the inputter 7. The medical image obtainer 58 obtains the medical image 56 and transfers the medical image 56 to the image processor 59. The medical image 56 transferred to the image processor 59 is combined with the image operation information 55, and the medical image 56 with the image process applied is combined. Then, the image processor 59 transfers the combined medical image 56 to the image display controller 16 in the mobile terminal 1 and the image display controller 36 in the hospital terminal 3.

Here, according to the present embodiment, the instruction information (sound) 51 is linked to the image operation information 55 or the instruction information (text) 53 is linked to the image operation information 55. However, since the medical image 56 is combined with the image operation information 55, the combined medical image 56 is linked with the sound data as a result.

According to the present embodiment, only the spoken contents by the speaker in the sound data is obtained. When all of the sound data including noise is obtained, all of the sound data is linked with the combined medical image 56.

According to the present embodiment, only the image operation information 55 is obtained in the mobile terminal 1, but the image data including the medical image 56 and the image operation information 55 combined from the beginning can be obtained.

According to the present embodiment, the image operation information 55 is obtained in a still image state, but this can be obtained in a moving image state. That is, according to the present embodiment, the image operation information 55 is obtained as a still image as a result of adding the annotation a. Alternatively, the moving image while adding the annotation a can be obtained.

According to the present embodiment, the medical image 56 is a still image but this may be a moving image. Even if the medical image 56 is a moving image, the moving medical image and the image operation information 55 can be obtained separately and then combined as in the present embodiment. Alternatively, the moving medical image and the image operation information 55 may be obtained in a state combined from the beginning.

According to the present embodiment, the sound data and the image operation information 55 are stored in the mobile terminal 1 and the hospital terminal 3, and the instruction information (sound) 51 is linked to the image operation information 55 in the server 5, but the present invention is not limited to the above. For example, all of the processes up to the linking of the instruction information (sound) 51 and the image operation information 55 may be performed in the terminal, and only the process of storing the final data used later for confirming the instruction may be performed in the server 5.

According to the present embodiment, the mobile terminal 1 is connected to the hospital terminal 3 through the server 5, but the terminals may be connected directly through an internet connection without using the server 5.

The above is the procedure flow by the server 5.

According to the viewer process, the sound process, and the server process as described above, the contents of the conversation between the medical specialist and the medical doctor in the hospital while viewing the image are stored. With this, the contents regarding the medical treatment instructed by the medical specialist can be stored.

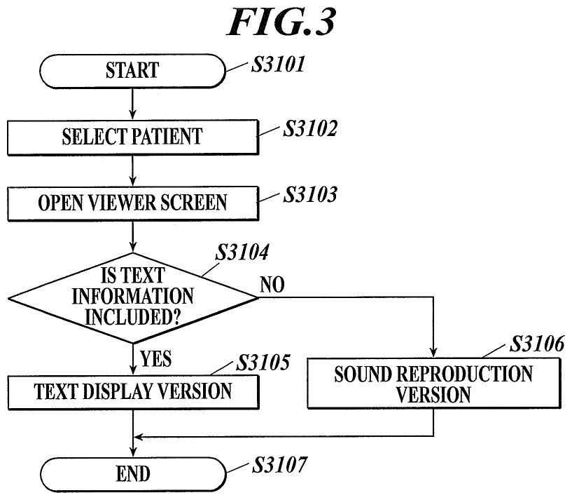

Next, the method to confirm the medial instruction on the terminal using the information obtained by the viewer process, the sound process, and the server process is described with reference to the instruction confirmation flow shown in FIG. 3.

The instruction can be confirmed using any of the mobile terminal 1 or the hospital terminal 3. The present embodiment describes confirming the instruction using the hospital terminal 3.

The instruction confirmation flow starts as step S3101. The instruction confirmation starts by pressing the start button (not shown).

Next, the patient for which the instruction is confirmed is selected as step S3102. The upper side of FIG. 2D is an example of the list screen Li. The line La in the list screen Li showing the patient to be examined is touched, pressed or double-clicked to make the selection. When the patient is selected, the viewer screen V as shown in the lower side of FIG. 2D opens (step S3103).

Next, it is determined whether the user selected to include the instruction information (text) 53 as the method to output the instruction information. This is to be step S3104. When it is determined that the user selected to include the display of the instruction information (text) 53 (Yes in step S3104), the process advances to step S3105. When it is determined that the user selected not to include the display of the instruction information (text) 53 (No in step S3104), the process advances to step S3106.

When the process advances to step S3105, the text display version is selected, and the instruction information (text) 53 is displayed with the medical image 56 combined by the image processor 59. Here, the instruction/image operation relation controller 34 controls the display so that the instruction information (text) 53 and the image operation information 55 linked to each other by the instruction/image operation linking unit 60 are displayed in coordination with each other. Based on the control by the instruction/image operation relation controller 34, the instruction information display controller 35 displays the instruction information (text) 53 on the display/operating unit 3a of the hospital terminal 3, and the image display controller 36 displays the combined medical image 56 on the display/operating unit 3a of the hospital terminal 3.

When the process advances to step S3106, the sound reproduction version is selected, and the instruction information (sound) 51 and the image combined by the image processor 59 are displayed. Here, the instruction/image operation relation controller 34 controls the display so that the instruction information (sound) 51 and the image operation information 55 linked to each other by the instruction/image operation relation linking unit 60 are displayed in coordination with each other. Based on the control by the instruction/image operation relation controller 34, the instruction information display controller 35 displays the instruction information (sound) 51 on the display/operating unit 3a of the hospital terminal 3 and the image display controller 36 displays the combined medical image 56 on the display/operating unit 3a of the hospital terminal 3.

Here, the instruction of the medical specialist can be confirmed more accurately if both of the instruction information (text) 53 and the image operation information 55 or both of the instruction information (sound) 51 and the image operation information 55 are displayed. However, according to the present invention both of the instruction information (text) 53 and the image operation information 55 or both of the instruction information (sound) 51 and the image operation information 55 do not have to be displayed, and only either one may be displayed.

The specific method of displaying the text display version and the sound reproduction version is described later in the examples.

### Example 1

In the following example, the method of use is described according to the flow when the product employing the present invention is actually used. First, the operation flow to perform the sound input and the image operation using the mobile terminal 1 and the hospital terminal 3 is described, and then, the method of confirming the instruction is described. The flow of the sound input and the image operation describes one example, and two examples (example 1, example 2) are described regarding confirming the instruction.

[Sound Input/Image Operation Flow]

The present example assumes a situation in which the medical specialist at home uses the mobile terminal 1 and the duty doctor in the hospital uses the hospital terminal 3.

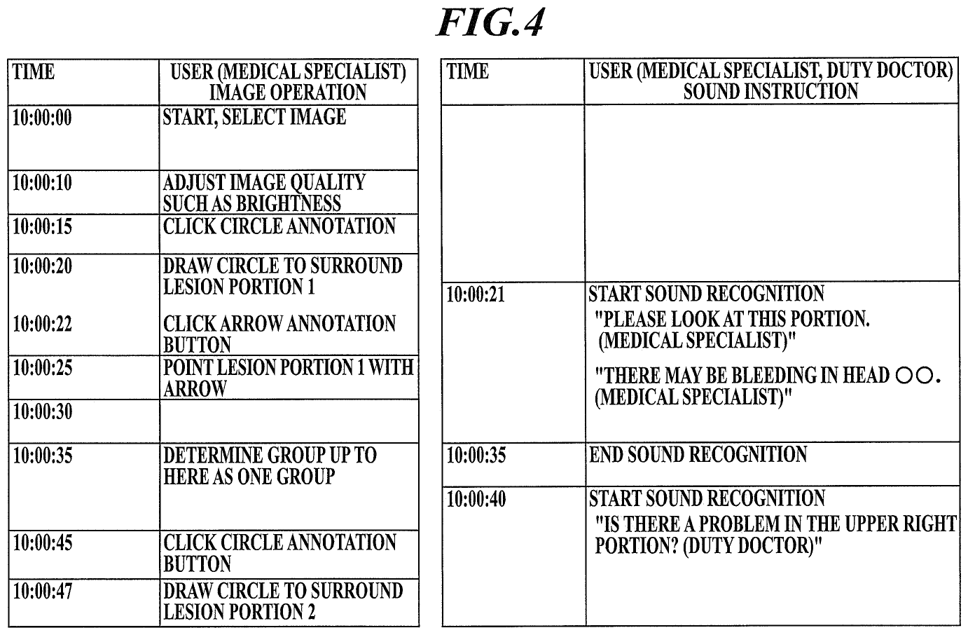

FIG. 4 shows the flow in time in the user (medical specialist and duty doctor) operation flow. The left table shows the operation when the user (medical specialist) uses the mobile terminal 1, and the right table shows the sound instruction of the user (medical specialist and duty doctor).

First, the medical specialist performs the start operation and the medical image selection of the patient to be diagnosed at 10:00:00 (showing 10 o'clock, that is hour 10, minute 0, second 0).

According to the present example, the medical image 56 is a still image such as a CT image. At the same time, the duty doctor starts the operation and selects the same patient as the patient selected by the medical specialist. Alternatively, the duty doctor may start operation before 10:00:00, and the screen may be shared with the mobile terminal 1 used by the medical specialist. The screen of the mobile terminal 1 is shared with the duty doctor. The present example assumes the state sharing the screen.

Next, at 10:00:10, the medical specialist enlarges the target portion of the medical image 56 and adjusts the tone to make a suitable observation.

Also at this point, an expansion process of the medical image 56 can be performed.

Next, at 10:00:15, the medical specialist clicks the circle annotation button B9. The group ID=1 is attached when the specific image operation, here the operation to add the annotation a, is performed because this is the first operation. When the image operation such as the tone process and the expansion display is performed up to this point, the same group ID=1 is attached to the image operation. Here, the mobile terminal 1 transfers the information of the image operation to the server 5. This information is transferred to the hospital terminal 3, and the duty doctor confirms the image operation contents displayed on the hospital terminal 3.

When the tone process is performed before 10:00:15, the same group ID is attached to these image operations. For example, when the tone process and the expansion process are performed at 10:00:12, the group ID=1 is attached to the image operations (tone process and expansion process) and all of the image processes clicking the circle annotation button B9 performed at 10:00:15. That is, the medical image 56 is stored in a state with all of the image operations performed.

Next, at 10:00:20, the medical specialist draws a circular annotation a to surround the desired portion (lesion site Le1) of the medical image 56. Such operation is performed before 10 seconds pass from the previous image operation in which the group ID=1 is attached at 10:00:15. Therefore, the group ID=1 is attached without updating the group ID. Here, similar to when the previous group ID=1 is attached, the image operation information 55 is transferred to the server 5 and the hospital terminal 3, and the duty doctor confirms the displayed image operation contents. From here after, the similar process is performed each time the group ID is attached, but the description is omitted.

Next, at 10:00:21, the sound recognition starts when the medical specialist starts speaking. The spoken contents by the medical specialist is obtained by the mobile terminal 1 and transferred to the server 5. The spoken contents by the duty doctor is obtained by the hospital terminal 3 and transferred to the server 5.

Next, at 10:00:22, the medical specialist clicks the arrow annotation button B9. The time that passed is 10 seconds or less from the image operation at 10:00:20, and the group ID=1 is attached without updating the group ID.

Next, at 10:00:25, the medical specialist points the lesion site Le1 with the arrow annotation a. This is before 10 seconds pass from the click of the arrow annotation button B9 at 10:00:22. Therefore, the group ID=1 is attached without updating the group ID.

Then, when 10 seconds pass from the arrow operation at 10:00:25 and it becomes 10:00:35, the group of image operation up to this point is determined to be one group and the group ID (=1) is stored. That is, when the image operation is performed next time, the group ID is updated.

Simultaneously with the determination of the group of image operations being one group at 10:00:35, the sound data is also determined to be one and the sound data is stored.

It may be difficult to define the standard to determine the break of the sound. Therefore, preferably, the point that the group ID of the image operation is updated is to be the reference, and the group ID of the sound is updated at the same time. Alternatively, the features of the sound data can be used to update the group ID of the sound data separately from the image operation.

Further, at this point, the image operation information 55 and the sound data are linked by the group ID (=1). That is, all of the image operation information 55 with group ID=1 attached are linked with the sound data with group ID=1 attached.

According to the present example, the adding of the annotation a is to be the start, and for example, as the start of the extracting operation by the user, all of the image operation information which is performed before the extraction operation and in which the same group ID is attached can be linked with the sound data obtained at the time that the image operation was performed.

The above-described extraction operation by the user is not limited and any operation can be made. Examples include pressing the snapshot button B11.

According to the present example, the sound recognition starts at the same time as when the speaking starts, but the timing of the start is not limited. For example, the sound recognition can start at the same timing as performing the image operation such as adding the annotation a.

According to the present example, the group ID is updated in the image operation information 55 in coordination with the sound data, but as described above, the group ID can be updated separately. In this case, for example, even if the same group ID is attached to the sound data and the image operation information 55, there may be a large difference in the time. In this case, the sound data and the image operation information 55 can be linked by matching the group ID. That is, the group ID is attached to show a relation between the sound data and the image operation information 55. The relation other than “time” may be, for example, the combination of the type of annotation a and specific spoken words, and this may be determined by the user in advance.

Next, at 10:00:40, the sound recognition starts when the duty doctor starts speaking. The hospital terminal 3 obtains the spoken contents by the duty doctor and transfers the above to the server 5.

The group ID=2 is attached to the spoken contents.

Next, at 10:00:45, the medical specialist clicks the circle annotation button B9. The previous image operation is performed at 10:00:25, and more than 10 seconds passed. Therefore, the group ID=2 is newly attached and stored.

Next, at 10:00:47, the medical specialist draws a circular annotation a to surround a predetermined portion (lesion site Le2) of the medical image 56. The group ID=2 is attached to such operation.

The operation after 10:00:47 is not shown in FIG. 4. However, similar to the flow of 10:00:00 to 10:00:35, the image operation information 55 is linked to the sound data by the group ID.

According to the present example, whether to update the group ID is determined when the annotation a is attached. Other than the method in which the system makes a judgment automatically, the user may update the group ID by clearly pressing the snapshot button B11 at the point when the related description ends and before the next related description starts.

The input of sound, storage of the image operation and the link between the sound data and the image operation information 55 are performed as described above.

[Instruction Confirmation Operation Flow]

Next, the instruction confirmation operation flow is described. As the method to display the instruction confirmation, the two examples, example 1 and example 2 are described.

### Example 1

The text display version is described in example 1. The text display version displays the instruction information (text) 53 obtained by converting the sound data to text, and the combined image obtained by combining the medical image 56 with the image operation information 55 (corresponding to the operation information combining image data or the operation information combining moving image data in the present invention). The instruction information (text) 53 and the image operation information 55 are displayed in coordination with each other with emphasis.

FIG. 5A shows a screen display example of the text display version. The instruction information (text) 53 and the combined image are displayed on the display/operating unit 3a of the hospital terminal 3.

The following are displayed in the example shown in FIG. 5A. The patient information, the group of various buttons (screen switching button B3, various operation buttons B4 to B11 including annotation button B9, and snapshot button B11, and end button B12), and thumbnail images are displayed in the upper portion of the viewer screen V. The medical image 56 with the annotation a attached is displayed in the image display area Va on the right half of the screen. A sound data timeline Tv is displayed as a vertical line to the left from the center. The instruction information (text) 53 is displayed in the instruction information display area Vb at the left side of the sound data timeline Tv. The image data timeline Ti is displayed at the lower portion of the screen.

Described in detail below, the “time series sound data in the time region with high importance” regarding the present invention corresponds to B1 and B2 in FIG. 5A (B1 and B2 in FIG. 5B). The “operation information combining data or the operation information combining moving image data in the time region with high importance” corresponds to C1 and C2 in FIG. 5A (C1 and C2 in FIG. 5B).

According to the present embodiment “time series sound data in the time region with high importance” according to the present invention is not limited to B1 and B2 in FIG. 5A, and can be any region in the time series sound data. Further, the user may be able to specify or correct the time region with the high importance later.

The same conditions as the time series sound data apply for the “operation information combined data or the operation information combined moving image data in the time region with high importance” according to the present invention.

The example 1 assumes adding the annotation twice (different group ID attached) as the image operation, and when the instruction is confirmed, the final image, that is, the image with two annotations a added is displayed.

Two annotations a are added to the medical image 56, and the numbers A1 and A2 are attached to each. A1 and A2 apply to the “image data in the time region with high importance” according to the present invention.

The numbers C1 and C2 are attached to the image data timeline Ti. C1 and C2 each correspond to A1 and A2. The image timeline Ti shows the time scale when the image operation is performed (start to end is shown from left to right). This shows that the annotation a is attached in the order from C1 to C2 in a time series, that is, in the order from A1 to A2.

The numbers B1 and B2 are attached to the sound data timeline Tv. B1 and B2 correspond to A1 and A2 as described above.

When the text of the instruction information (text) 53 is long and the entire instruction information (text) 53 cannot be displayed on the same screen, part of the instruction information (text) 53 is displayed and the instruction information (text) 53 before and after the displayed portion can be displayed by scrolling the screen vertically.

Here, for example, when the portion of C1 which is the time region with high importance in the image data timeline Ti is touched, C1 is brightly displayed with emphasis, and C2 is displayed dark so as not to stand out. Similar display is shown for A1 and A2, and B1 and B2. Further, the text of the portion which corresponds to B1 in the instruction information display area Vb is also brightly displayed with emphasis. With this, when the annotation a with the number A1 is added, the contents of the instruction by the medical specialist can be understood immediately, and it is possible to accurately understand the contents of the instruction regarding the medical treatment made by the medical specialist.

C1 is touched in the above description but the same display state is generated when A1 or B1 is touched. That is, regardless of whether the sound data or the image data is touched, both are displayed with emphasis in coordination with each other.

According to the present example, both the sound data timeline Tv and the image data timeline Ti are displayed on the screen but only either one may be displayed. Even if both are displayed, the user may perform an operation so as not to display one of the above.

According to the present example, the sound data and the image data are output in the form of a display on the display screen (display/operating unit 1a, 3a), but the output does not always have to be displayed. For example, as described in the example 2 described below, the output may be in a form of reproducing sound.

According to the present example, the medical image 56 is displayed in the final state with the annotation a added to the medical image 56 combined on the medical image 56 (final state) but the display of the added annotation a can be turned off. In this case, for example, when B1 or C1 is touched, the annotation a which is not displayed and which corresponds to A1 may be displayed.

For example, when any region in the image data timeline Ti or the sound data timeline Tv is touched, the sound data timeline Tv reacts in coordination so that the instruction information (text) 53 at the touched time may be displayed, and further, the image reflecting the image operation information 55 at the touched time may be displayed.

According to the present example, the time region with high importance in the sound data is B1 and B2, that is, the time that the speaker is speaking, but the time region with high importance is not limited to the above. For example, instead of the entire time duration that the speaker is speaking, the time that the speaker is speaking an important word specified in advance or the time that a specific speaker (medical specialist, etc.) is speaking can be set as the time with high importance.

As the method to extract the important words specified in advance, there is a method to automatically extract the important words from the text after the sound data is converted to text. However, the method is not limited to the above.

Preferably, the important words are words related to the medical instruction, for example, lungs, brain, MRI, and the like. Different words can be specified as the important words for each medical specialist.

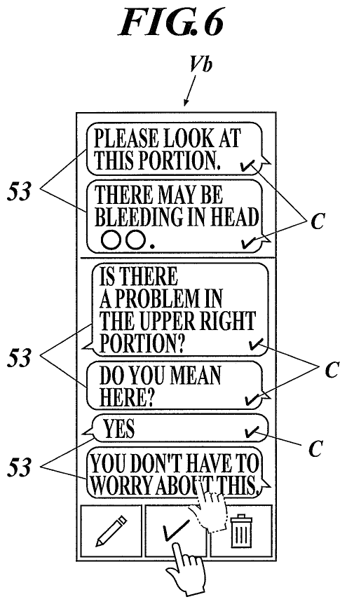

As shown in FIG. 6, when the instruction information (text) 53 is confirmed, a check may be attached to portions which are confirmed. For example, when the duty doctor confirms the instruction and the instruction information (text) 53 is long, confirming the contents already confirmed again becomes a burden. Therefore, as shown in FIG. 6, by attaching a checkmark C to the instruction information (text) 53 already confirmed, the instruction can be confirmed efficiently.

### Example 2

The sound reproduction version is described in example 2. In the sound reproduction version, the instruction information (text) 53 obtained by converting the sound data to text is not displayed. The sound data and the combined image obtained by combining the medical image 56 and the image operation information 55 are displayed. The sound data and the image operation information 55 are displayed in coordination with each other with emphasis.

FIG. 5B shows the screen display example of the sound reproduction version. The sound data and the combined image are displayed on the display screen of the hospital terminal 3.

In the example shown in FIG. 5B, the following items are displayed similar to FIG. 5A. The patient information, the group of various buttons (various operation buttons B4 to B11 including screen switching button B3, annotation button B9, and snapshot button B11, and end button B12), and thumbnail images are displayed in the upper portion of the viewer screen V. The medical image 56 with the annotation a attached is displayed in the image display area Va covering the majority of the screen. A sound data timeline Tv is displayed as a vertical line to the left of the screen. The image data timeline Ti is displayed at the lower portion of the screen. Further, various sound buttons B13 to B19 including the reproduction button B17 are displayed in the lower portion of the screen, and the sound data can be reproduced.

Similar to the example 1, example 2 also assumes an example in which the annotation a is added twice as the image operation (different group ID is added), but the added annotation a is not displayed in FIG. 5B.

B1 and B2 and C1 and C2 in FIG. 5B have the similar meaning as in the example 1 (FIG. 5A). Therefore, the operation of emphasizing the display in coordination with each other is similar to the example 1.

The example 2 is different from the example 1 in that, instead of displaying the instruction information (text) 53, the sound is reproduced. For example, when the portion of C1 is touched in the image data timeline Ti, the portion of B1 in the sound data timeline Tv is selected in coordination with the above. When the reproduction button B17 is pressed, the sound data in the region of B1 is reproduced. At the same time, the image data in C1 is also reflected, and the annotation a corresponding to C1 is displayed on the medical image 56. With this, similar to the example 1, the medical instruction by the medical specialist can be understood accurately.

Similar to example 1, in example 2 also both the sound data timeline Tv and the image data timeline Ti are displayed on the screen, but only either one of the above may be displayed. Even if both are displayed, the user may perform an operation so as not to display one of the above.

The example 2 may be similar to the example 1 in the other points also.

According to the above description, an HDD is used as the computer-readable medium including the program regarding the present invention, but the example is not limited to the above. Other computer-readable mediums such as a nonvolatile semiconductor memory, or a portable recording medium such as a CD-ROM may be applied. A carrier wave may be employed as the medium to provide the data of the program regarding the present invention through communication lines.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-118370 filed on Jun. 22, 2018 is incorporated herein by reference in its entirety.

What is claimed is:

1. A non-transitory computer-readable storage medium having a program stored thereon for controlling a computer to perform:
obtaining time series sound data which includes spoken contents by a speaker;
obtaining operation information combined image data or operation information combined moving image data including a medical image and image operation information which includes contents of an image operation performed on the medical image;
extracting the time series sound data in a time region with high importance from the time series sound data;
extracting the operation information combined image data or the operation information combined moving image data in the time region with high importance from the operation information combined image data or the operation information combined moving image data; and
outputting at least one of the time series sound data in the time region with high importance and the operation information combined image data or the operation information combined moving image data in the time region with the high importance.

2. The storage medium according to claim 1, wherein in the sound data obtaining, the time series sound data when the speaker is speaking is obtained from the time series sound data as the time series sound data in the time region with the high importance.

3. The storage medium according to claim 2, wherein in the sound data obtaining, the time series sound data in which the speaker can be identified is obtained as the time series sound data.

4. The storage medium according to claim 2, wherein in the sound data obtaining, the time series sound data in which the speaker is speaking an important word specified in advance is obtained from the time series sound data as the time series sound data in the time region with the high importance.

5. The storage medium according to claim 1, wherein in the image/moving image data extracting, the operation information combined image data or the operation information combined moving image data when the image operation is performed on the medical image is obtained from the operation information combined image data or the operation information combined moving image data as the operation information combined image data or the operation information combined moving image data in the time region with the high importance.

6. The storage medium according to claim 5, wherein the image operation is adding annotation to the medical image.

7. The storage medium according to claim 1, wherein the program further includes linking in a time series the time series sound data with the operation information combined image data or the operation information combined moving image data.

8. The storage medium according to claim 7, wherein in the sound/image linking, the time series sound data in the time region with the high importance is linked in the time series with the operation information combined image data or the operation information combined moving image data in the time region with the high importance.

9. The storage medium according to claim 7, wherein in the sound/image linking, the operation information combined image data or the operation information combined moving image data including all of the image operation information performed on the medical image at a point that the annotation is added to the medical image is linked in a time series with the sound data before the point that the annotation is added to the medical image.

10. The storage medium according to claim 7, wherein in the sound/image linking, the operation information combined image data or the operation information combined moving image data including all of the image operation information performed on the medical image at a point that the user performs extracting operation is linked in a time series with the sound data before the point that the user performs the extracting operation.

11. The storage medium according to claim 1, wherein in the data outputting, both the time series sound data in the time region with the high importance and the operation information combined image data or the operation information combined moving image data in the time region with the high importance are output.

12. The storage medium according to claim 11, wherein in the data outputting, when either one of the time series sound data in the time region with the high importance, or the operation information combined image data or the operation information combined moving image data in the time region with the high importance is selected, it is considered that both the one and the other are selected.

13. The storage medium according to claim 1, wherein in the data outputting, at least either one of the time series sound data in the time region with the high importance, or the operation information combined image data or the operation information combined moving image data in the time region with the high importance is displayed on a display.

14. The storage medium according to claim 1, wherein the program further includes,
converting the spoken contents into text; and
displaying the text on the display.

15. A medical instruction output method comprising:
obtaining time series sound data which includes spoken contents by a speaker;
obtaining operation information combined image data or operation information combined moving image data including a medical image and image operation information which includes contents of an image operation performed on the medical image;
extracting the time series sound data in a time region with high importance from the time series sound data;
extracting the operation information combined image data or the operation information combined moving image data in the time region with high importance from the operation information combined image data or the operation information combined moving image data; and
outputting at least one of the time series sound data in the time region with high importance and the operation information combined image data or the operation information combined moving image data in the time region with the high importance.

16. A medical instruction output apparatus comprising:
a hardware processor which performs,
obtaining time series sound data which includes spoken contents by a speaker;
obtaining operation information combined image data or operation information combined moving image data including a medical image and image operation information which includes contents of an image operation performed on the medical image;
extracting the time series sound data in a time region with high importance from the time series sound data;
extracting the operation information combined image data or the operation information combined moving image data in the time region with high importance from the operation information combined image data or the operation information combined moving image data; and
outputting at least one of the time series sound data in the time region with high importance and the operation information combined image data or the operation information combined moving image data in the time region with the high importance.

17. A medical instruction output system comprising:
a server including a hardware processor which performs,
obtaining time series sound data which includes spoken contents by a speaker;
obtaining operation information combined image data or operation information combined moving image data including a medical image and image operation information which includes contents of an image operation performed on the medical image;
extracting the time series sound data in a time region with high importance from the time series sound data; and
extracting the operation information combined image data or the operation information combined moving image data in the time region with high importance from the operation information combined image data or the operation information combined moving image data; and
a terminal which displays at least one of the time series sound data in the time region with high importance and the operation information combined image data or the operation information combined moving image data in the time region with the high importance.
